(19) United States
(12) Patent Application Publication
(10) Pub. No.: US 2006/0064034 A1
(43) Pub. Date: Mar. 23, 2006

(54) Midstream urination collection device

(76) Inventors: David McIntyre Stewart, Pittsburgh, PA (US); Suanne Craig Stewart, Pittsburgh, PA (US)

Correspondence Address: Reed Smith LLP, 435 Sixth Avenue, Pittsburg, PA, 15219-1886, US

(21) Appl. No.: 11/234,843

(22) Filed: Sep. 23, 2005

Related U.S. Application Data
(63) Continuation-in-part of application No. 10/949,621, filed on Sep. 23, 2004.
(60) Provisional application No. 60/650,947, filed on Feb. 7, 2005.

(51) Int. Cl. A61B 5/00

(52) U.S. Cl. 600/573; 600/574

(57) ABSTRACT

An apparatus for the sanitary and simple collection of mid-stream urine discharges from medical patients. Presently-preferred embodiments of the present invention include a collection chamber that is adapted to allow for the collection of a midstream portion of a urine discharge. By selectively allowing the initial portion of the urine discharge to escape a collection chamber, the embodiments of the present invention provide for the collection of the midstream portion of a urine discharge.

### CROSS-REFERENCE TO RELATED APPLICATIONS

This application claims priority to provisional patent application Ser. No. 60/650,947, filed Feb. 7, 2005, and continuation-in-part status from co-pending utility application Ser. No. 10/949,621, filed Sep. 23, 2004.

### BACKGROUND OF THE INVENTION

1. Field of the Invention

The present invention relates to apparatuses for the collection of the midstream portion of a urine discharge from a medical patient. The apparatus preferably allows the midstream portion of a urine discharge to be collected largely without contamination from, and independently of, an initial portion of a urine discharge.

2. Description of the Background

Collection of urine samples from medical patients for chemical analysis commonly occurs in numerous patient populations, including those with urinary tract infections, those undergoing chemotherapy, and those afflicted with chronic and debilitating illnesses. A common concern of the medical practitioner, however, is contamination of the urine sample by agents and organisms that may be present in the urinary tract or the adjacent anatomy.

The problem of contamination is even greater for female patients than for males because of the location of the urethral opening just superior to the vaginal opening. Secretions and other contaminants which are present in the vaginal area are particular concerns. It is therefore desirable that the urine sample is collected from a midstream portion of a urine void so that the urinary tract and other proximal areas can be “flushed” prior to the sample collection.

The simplest method for collecting a mid-stream urine specimen is to allow the urine flow to begin, wait a period of time, and then insert a collecting vessel into the urine stream. Since it is difficult to stop the flow after it has begun, it is also difficult to catch the mid-stream sample without soiling the hands and the outside of the collection container. In addition, the patient may be unable to assess accurately when to begin collection. If, on the other hand, a second person is required to assist or begin collection, it presents an awkward and potentially embarrassing situation for the medical patient.

While numerous mid-stream urination collection devices are described in the prior art, their operation is cumbersome and often results in the spilling of urine or other undesirable outcomes.

There has been a long standing need in the medical community for a cost effective urination collection device that collects a midstream urine sample that is simple to use and avoids contamination by the initial portion of the urine stream, while at the same time preserving the dignity of the patient. In addition, the device should also provide for the collection of multiple samples in a sanitary and non-contaminating manner.

### SUMMARY OF THE INVENTION

It is an object of the present invention to provide apparatuses that are useful for the collection of the isolated midstream portion of a urine discharge, free from contamination by the forestream portion (i.e. the initial void) of a urine discharge. The apparatuses are particularly suitable for use with adult female patients, but also have applications in the adult male, geriatric, and pediatric patient populations as well. As the apparatuses of the present invention are to be used for collection of urine samples for testing, the apparatus would preferably be sterilized prior to use with a patient.

In accordance with the present invention, the urine discharge is collected from a patient into a chamber via a collection funnel. The collection funnel discharges into the chamber at a funnel outlet. The chamber preferably contains a chamber outlet at its bottom that further includes a taper or valve that is adapted to regulate or control the flow of collected urine from the chamber. At the top of the chamber, and preferably above (i.e., more proximal than) the funnel outlet, a sample collection discharge port is preferably located. The sample collection discharge port is operably connected to at least one sample collection vessel.

The various embodiments of the present invention may be constructed of numerous types of materials. In particular, plastics including polyethylene and polystyrene are presently preferred. In some embodiments, the apparatuses are constructed of plastics that may safely be autoclaved while retaining structural integrity. That property would allow the urination collection devices of the present invention to be reused after contamination with a urine sample. Alternatively, the apparatuses of the present invention may be constructed to be used once and then discarded.

During operation of certain presently-preferred embodiments of the present invention, the patient begins to discharge urine into a collection funnel at the top of the apparatus. The urine drains to a collection chamber, where the initial portion of the urine stream escapes out of the bottom of the chamber via a tapered section. By limiting further flow, the tapered section causes the mid- and late-stream portions of the urine discharge to accumulate in the chamber. The level of urine in the chamber eventually rises to allow the fluid to flow through a discharge port that connects to a collection tube, where the urine sample is collected into a collection container for subsequent analysis. In some presently-preferred embodiments, the collection chamber is constructed of a transparent material to allow for the direct observation of the accumulation of urine by the patient or medical practitioner.

In other presently-preferred embodiments of the invention, the urine discharge is collected from a patient into a collection chamber via a collection funnel. In those embodiments, the collection chamber includes a valve at the bottom of the chamber that may be opened to allow the forestream portion of the urine discharge to vacate the chamber. The valve may then be closed (either partially or completely) allowing the chamber to fill with the remainder of the urine discharge. Preferably, the level of urine in the chamber rises to allow the fluid to flow through a port into a connecting tube that terminates at a collection tube, where the urine sample is collected.

In other presently-preferred embodiments, the geometry of the chamber is adapted so as to allow the initial portion of the urine discharge to flow to be isolated from the midstream portion of the urine discharge using a sliding barrier. Such embodiments also allow the patient to easily and sanitarily collect midstream urine discharge.

In other presently-preferred embodiments, the invention includes an additional tube that allows air to escape from the collection tube as it fills with urine.

### BRIEF DESCRIPTION OF THE DRAWINGS

For the present invention to be clearly understood and readily practiced, the present invention will be described in conjunction with the following figures, wherein like reference characters designate the same or similar elements, which figures are incorporated into and constitute a part of the specification, wherein:

FIG. 1 shows a first presently-preferred embodiment of the present invention;

FIG. 2 depicts a presently-preferred embodiment of the present invention which is adapted to include multiple collection vessels;

FIG. 3 shows a presently-preferred embodiment of the present invention, in which a pinch valve is employed;

FIG. 4 displays a presently-preferred embodiment of the present invention having an alternative geometry for the chamber;

FIG. 5 shows a presently-preferred embodiment of the present invention having an alternative geometry for the collection funnel;

FIG. 6 displays a presently-preferred embodiment of the present invention that includes a turn valve;

FIG. 7 shows a presently-preferred embodiment of the present invention that is adapted to include multiple collection tubes and a turn valve;

FIG. 8 displays a presently-preferred embodiment of the present invention including a turn valve and having an alternative geometry for the collection funnel;

FIG. 9 displays a presently-preferred embodiment of the present invention including a tube that allows air to escape the collection tube as the collection tube fills with urine.

### DETAILED DESCRIPTION OF THE INVENTION

It is to be understood that the figures and descriptions of the present invention have been simplified to illustrate elements that are relevant for a clear understanding of the invention. The detailed description will be provided hereinbelow with reference to the attached drawings.

The present invention preferably provides an apparatus for the sanitary and simple collection of mid-stream urine discharges from medical patients. The device is particularly useful for adult male and female patients, as well as geriatric and pediatric populations. The device may be held by the patient during urine collection or maintained in place through the use of a harness thus allowing for unattended collection of urine samples.

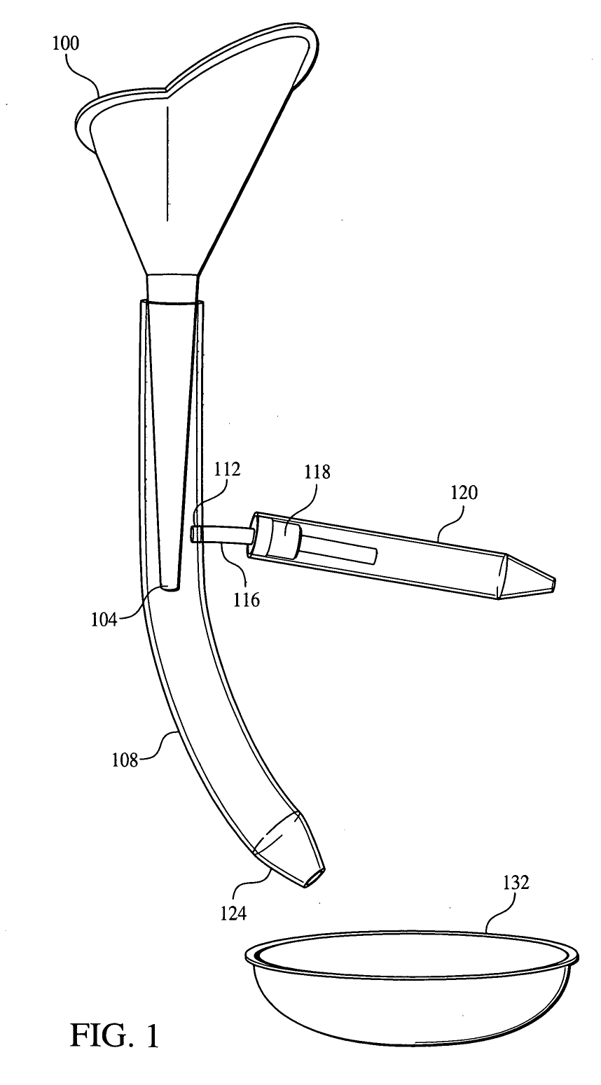

A presently-preferred embodiment of the present invention is displayed in FIG. 1. The apparatus in FIG. 1 includes a collection funnel 100 into which the patient urinates. The collection funnel 100 may be elliptical, as shown, or may be adapted to any other geometry (i.e. shape, size, anatomical configuration) that allows the collection of urine from a patient. In some embodiments, the proximal portion of the collection funnel 100 may include padding so that it may be placed comfortably against the genital region of a female patient.

The collection funnel 100 leads to a funnel outlet 104 which discharges into a chamber 108. While a cylindrical geometry for the chamber 108 is displayed in FIG. 1, the specific geometry of the chamber 108 is not important and various shapes including cubic and a flexible bag are considered within the scope of the present invention. At its distal aspect, the chamber 108 preferably terminates at a tapered restriction point 124. Within the context of the present invention, the term “proximal” will refer to the aspect of the apparatus or element located close to the patient, i.e., the top of the funnel. The term “distal” will refer to the aspect of the apparatus or element located farther from the patient.

The tapered restriction point 124 opens into a reservoir 132. The reservoir 132 may be closed container or an open container such as a bed pan, as shown in FIG. 1. The reservoir 132 is preferably of sufficient size to contain even a large urine discharge.

A port 112 is located at the proximal aspect of the chamber 108. The port 112 should be sufficiently large to permit the flow of fluids such as urine. The port 112 is preferably located above (i.e., more proximal than) the funnel outlet 104 and is adapted to fit a connecting tube 116 snugly. The connecting tube 116 also preferably fits snugly into a stopper 118 that seals a collection tube 120.

The apparatus displayed in FIG. 1 preferably operates in the following manner. The patient holds the collection funnel 100 up to his or her genitalia. The patient may be standing, sitting at an angle, or sitting over a toilet. Preferably, the apparatus resides below the genitalia of the patient. As the patient voids into the collection funnel, the initial portion of the urine discharge will flow through the chamber 108 and out the tapered restriction point 124. Because the tapered restriction 124 will restrict large flows of fluid, urine will accumulate in the chamber 108 under sustained urine flow. The chamber 108 eventually fills to the port 112. Urine then flows down the connecting tube 116 to the collection tube 120. After a sufficient urine sample was collected, the urine would drain from the chamber 108 into the reservoir 132 through the tapered restriction point 124.

One of skill in the art will recognize that the volume of the chamber may be adjusted to match the clinical population that is being served. For example, geriatric patients or pediatric patients would likely require a smaller chamber. The chamber size should be chosen so as to still allow the filling of the chamber following the disposal of the initial portion of the urinary discharge. In addition, the diameter and degree of taper of the tapered restriction point may be altered to adjust the amount of the initial discharge that is allowed to escape. The tapered restriction point may also be altered to adjust for the individualized rates of urine discharge. It would be routine for one of skill in the art to determine the specific parameters that would work best for each individual patient.

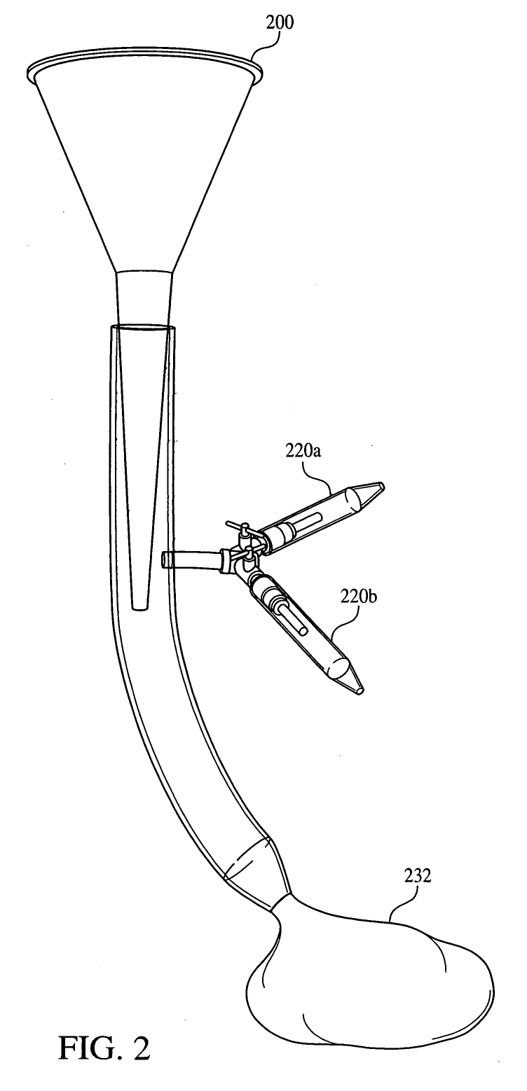

One of skill in the art will recognize that many variations may be employed in implementation of the present invention. For example, FIG. 2 displays a presently-preferred embodiment of the present invention in which the reservoir 232 may be a bag sufficient to contain the entire urinary discharge. In addition, the geometry of the collection funnel 200 may vary to suit individual patients' preferences. In this embodiment, the collection funnel 200 is circular in shape. Also contemplated within the scope of the present invention are apparatuses that include multiple collection tubes 220a 220b. That embodiment allows multiple, independent samples to be collected. In some of the preferred embodiments, the collection tubes 220a 220b may contain chemical reagents useful in diagnostic tests. In those circumstances, it may be desired to maintain independence among the various different urine samples. While this embodiment displays only two collection tubes 220a 220b, embodiments with a plurality of collection tubes are considered within the scope of the present invention.

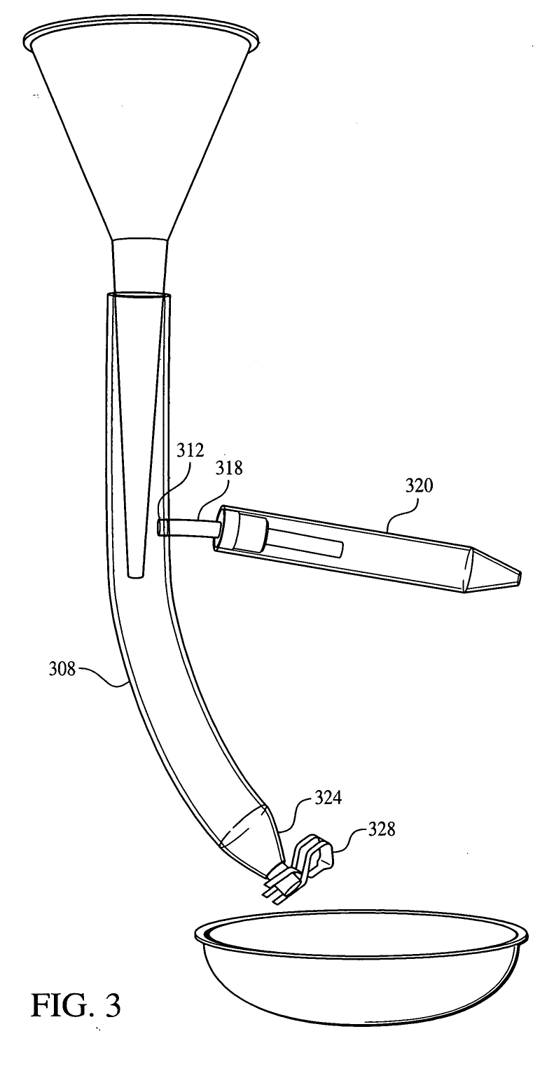

FIG. 3 displays another presently-preferred embodiment of the present invention in which a pinch valve 328 is employed at the bottom of the chamber 308. The pinch valve 328 preferably is open when squeezed and is closed when released. Prior to initiating urination, the patient may open the pinch valve 328 so that urine will flow through the pinch valve 328. After the initial portion of the urine flow has passed through the pinch valve 328, the patient may then release the pinch valve 328 allowing the chamber to fill 308. This results in the urine level rising to the level of the small port 312. The urine then drains through the connecting tube 318 into the collection tube 320. Also contemplated within the scope of the present invention are embodiments where the tubing at the taper 324 is flexible enough to be pinched by the fingers of the patient. In this manner, the patient may accomplish the same function as the pinch valve 328.

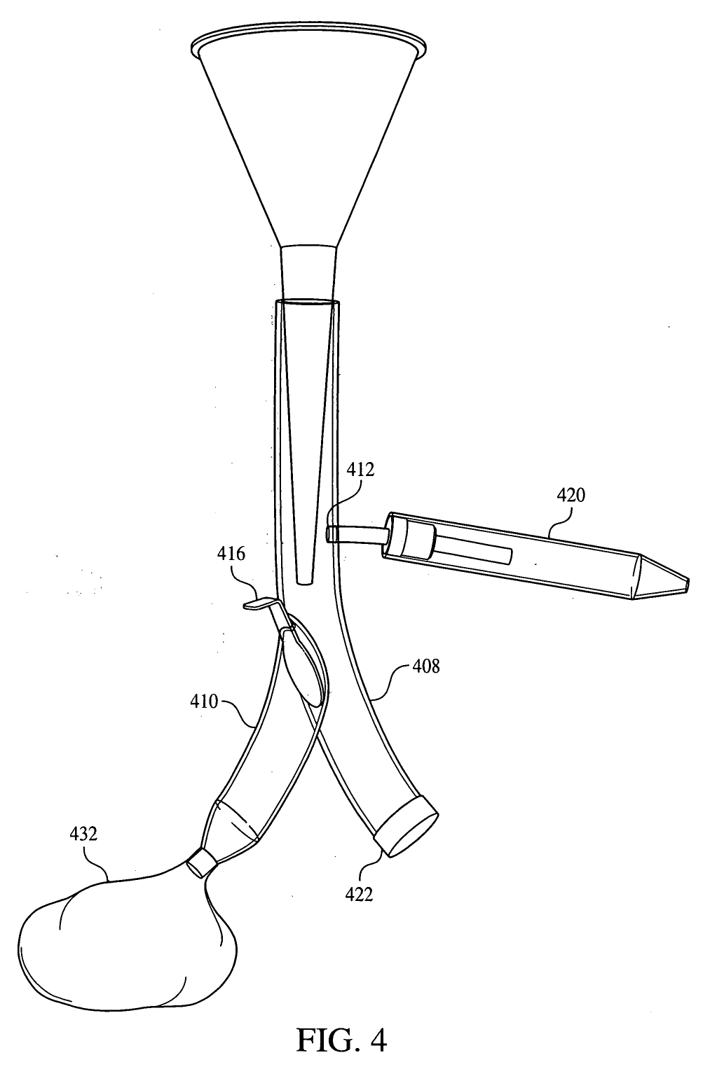

FIG. 4 displays another presently-preferred embodiment of the present invention. In this embodiment, a secondary chamber 410 is connected to the primary chamber 408. A sliding barrier 416 is preferably situated between the secondary chamber 410 and the primary chamber 408. The primary chamber 408 may be sealed at the bottom 422 so that urine will not drain from this chamber. The secondary chamber 410 is preferably connected to a reservoir 432. The secondary chamber 410 may also be connected to a container or simply be open so that it may drain to a toilet. During operation of the presently-preferred embodiment displayed in FIG. 4, the patient preferably adjusts the sliding barrier 416 so that urine may flow freely into the secondary chamber 410. Thereupon, the patient preferably orients the present invention so that the urine will, through gravity, flow into the secondary chamber 410 and drain into the reservoir 432. After the initial portion of the urine discharge has passed into the second chamber 410, the patient may then adjust the sliding barrier 416 so that urine fills the primary chamber 408. The urine then preferably fills the primary chamber 408 until it flows through the port 412 to the collection tube 420. When the collection tube is sufficiently full 420, the patient may adjust the sliding barrier 416 so that urine may flow into the secondary chamber 410 and into the reservoir 432.

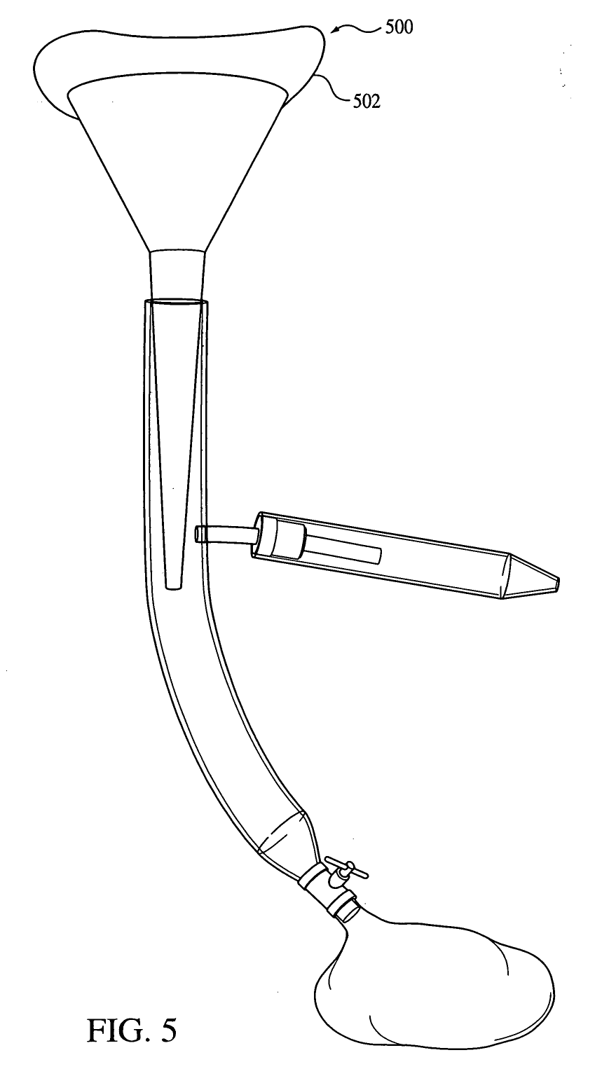

FIG. 5 displays a presently-preferred embodiment of the present invention in which the collection funnel 500 has been adapted to include a gel-like border 502 that may be placed against the medical patient's genitalia. This will provide the medical patient with an added level of comfort during use of the present invention.

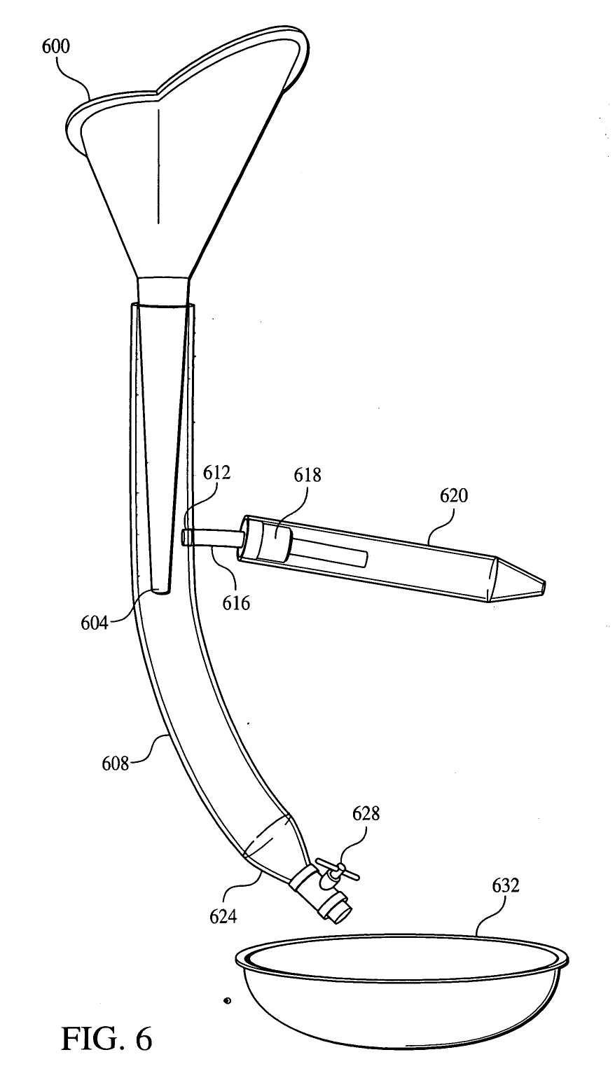

FIG. 6 shows a presently-preferred embodiment of the present invention that includes a turn valve 628 at the bottom of the collection chamber 608. The embodiment displayed in FIG. 6 preferably operates similarly to the embodiment shown in FIG. 1 and in the following manner. Prior to initiating urination, the patient opens the valve 628 so as to allow urine to flow out of the bottom of the chamber 608. The patient holds the collection funnel 600 up to his or her genitalia. The patient may be standing, sitting at an angle, or sitting over a toilet. As the patient voids into the collection funnel, the initial portion of the urine discharge will flow through the chamber 608 and out of the valve 628. After the first several seconds of urine discharge, the patient closes the valve 628 allowing the chamber 608 to fill with the midstream portion of urine. The chamber 608 eventually fills to the port 612. Urine would then flow down the connecting tube 616 to the collection tube 620. After a sufficient urine sample is collected, the patient could again open the valve 628 and the urine would drain from the chamber 608 into the reservoir 632.

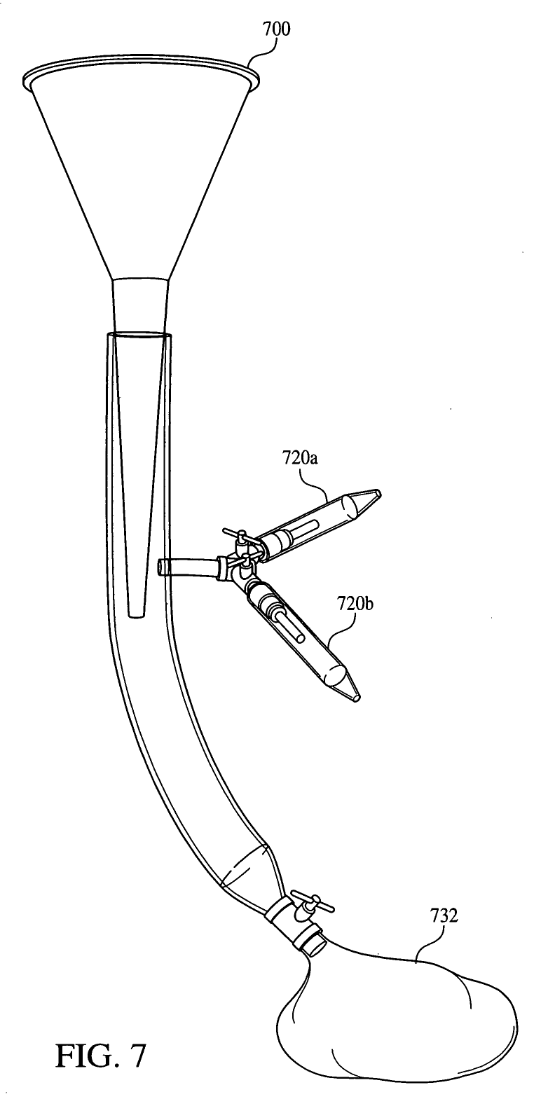

FIG. 7 displays an embodiment of the present invention that includes a twist valve at the bottom of the collection chamber and is adapted to allow for multiple collection tubes 720a 720b.

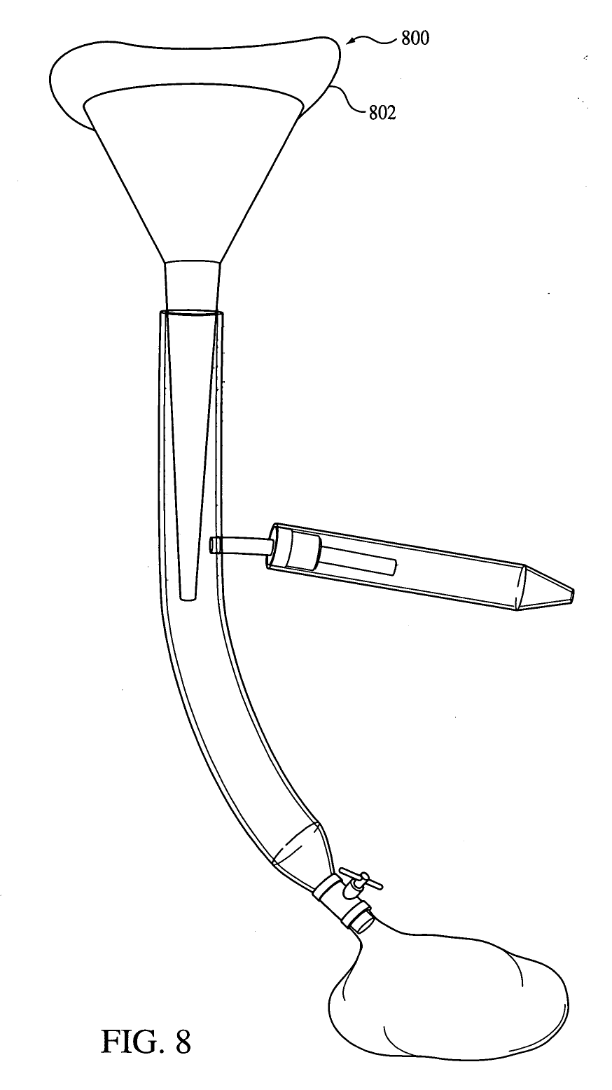

FIG. 8 shows an embodiment of the present invention that includes a turn valve. In this embodiment the collection funnel 800 has been adapted to include a gel-like border 802 that may be placed against the patient's genitalia.

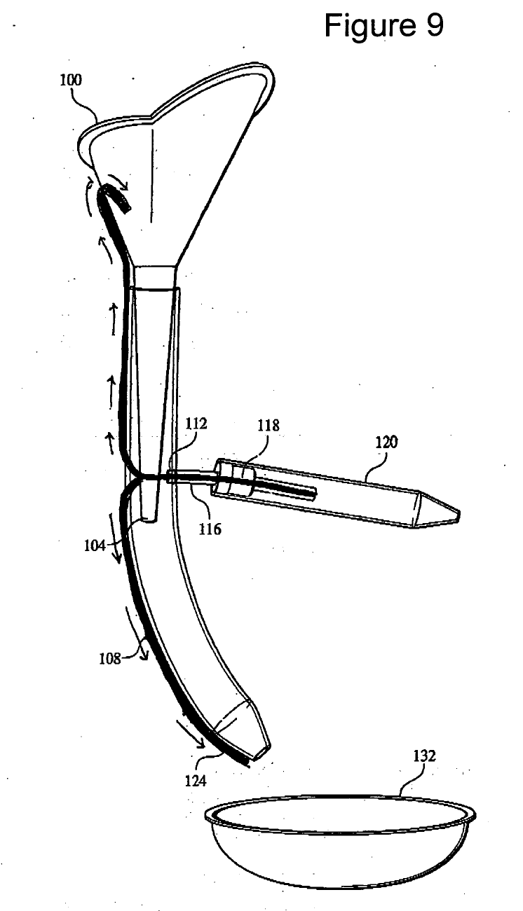

FIG. 9 shows an embodiment of the present invention that includes an additional tube that allows air to escape the collection tube as it fills with urine. In the presently-preferred embodiment, a small tube is adapted to fit into the connecting tube. The small tube then runs away from the collection tube and either to the collection funnel or to the reservoir. Both configurations are shown in FIG. 9.

The embodiment shown in FIG. 9 operates similarly to the embodiment shown in FIG. 1. As the patient voids into the collection funnel, the initial portion of the urine discharge will flow through the chamber and out the tapered restriction point. Because the tapered restriction will restrict large flows of fluid, urine will accumulate in the chamber under sustained urine flow. The chamber eventually fills to the port. Urine then flows down the connecting tube to the collection tube. As urine fills the collection tube, air will be allowed to escape the collection tube via the small tube that is located within the connecting tube. Once the collection tube has filled with urine, any excess urine may escape the collection tube via the small tube. Any overflow urine that enters the small tube would then discharge to either the reservoir or the collection funnel, depending on the configuration that was employed. The embodiment displayed in FIG. 9 has the added benefit of allowing the practitioner to adjust the level of urine that remains in the collection tube.

One of skill in the art will recognize that there are many possible ways of allowing air to escape the collection tube. For example, a small tube may access the collection independently, rather than being displaced within the connecting tube. Alternatively, the stopper or lid of the collection tube may be fit with a valve that allows air to escape, but does not allow urine to pass through. The examples displayed in FIG. 9 are meant to be illustrative and not limit the manner in which the invention may be implemented.

Nothing in the above description is meant to limit the present invention to any specific materials, geometry, or orientation of elements. Many part/orientation substitutions are contemplated within the scope of the present invention and will be apparent to those skilled in the art. The embodiments described herein were presented by way of example only and should not be used to limit the scope of the invention.

Although the invention has been described in terms of particular embodiments in an application, one of ordinary skill in the art of design, in light of the teachings herein, can generate additional embodiments and modifications without departing from the spirit of, or exceeding the scope of, the claimed invention. Accordingly, it is understood that the drawings and the descriptions herein are proffered only to facilitate comprehension of the invention and should not be construed to limit the scope thereof.

1. A urination collection device for collecting a midstream portion of a urine discharge from a medical patient, comprising:
a collection funnel, wherein said collection funnel discharges into a chamber via a funnel outlet;
a tapered restriction point at the distal aspect of said chamber, wherein said tapered restriction point discharges to a reservoir;
a port, wherein said port is located above said funnel outlet;
a connecting tube, wherein said connecting tube snugly fits into said port;
at least one collection tube, wherein said at least one collection tube is operably connected to said chamber by said connecting tube; and
an overflow tube, wherein said overflow tube is adapted to fit into said collection tube, further wherein said overflow tube discharges to the collection funnel or the reservoir.

2. The urination collection device of claim 1, further comprising a multidirectional stopcock located between said connecting tube and said at least one collection tube.

3. The urination collection device of claim 2, further comprising two, three, or four collection tubes.

4. The urination collection device of claim 1, wherein said reservoir is selected from the group consisting of a bed pan, a toilet, and a collection bag.

5. The urination collection device of claim 1, further comprising a gel-like border that may be placed against said medical patient, wherein said gel-like border is connected to a rim of said collection funnel.

6. The urination collection device of claim 1, wherein said chamber is cylindrical.

7. The urination collection device of claim 1, wherein said chamber is transparent.

8. The urination collection device of claim 1, wherein said urination collection device is constructed of a material that may be autoclaved.

9. A urination collection device for collecting a midstream portion of a urine discharge from a medical patient, comprising:
a collection funnel, wherein said collection funnel discharges into a primary chamber via a funnel outlet, further wherein said primary chamber is sealed at the distal end;
a secondary chamber, wherein said secondary chamber is open at the distal end, further wherein said secondary chamber is operably connected at a proximal portion of said secondary chamber to said primary chamber;
a sliding barrier that is adapted to separate said first chamber from said second chamber;
a port in said primary chamber, wherein said port is located above said funnel outlet;
a connecting tube, wherein said connecting tube snugly fits into said port;
at least one collection tube, wherein said at least one collection tube is plugged at an open end by a stopper, wherein said connecting tube passes through said stopper, further wherein said connecting tube operably connects said primary chamber to said at least one collection tube, and
an overflow tube, wherein said overflow tube is adapted to fit into said collection tube, further wherein said overflow tube discharges to the collection funnel or the reservoir.

10. The urination collection device of claim 9, further comprising a multidirectional stopcock located between said connecting tube and said at least one collection tube.

11. The urination collection device of claim 9, further comprising two, three, or four collection tubes.

12. A urination collection device for collecting a midstream portion of a urine discharge from a medical patient, comprising:
a collection funnel, wherein said collection funnel discharges into a chamber via a funnel outlet;
a valve at the distal aspect of said chamber, wherein said valve allows said chamber to discharge to a reservoir;
a port in said chamber, wherein said port is located above said funnel outlet;
a connecting tube, wherein said connecting tube snugly fits into said port;
at least one collection tube, wherein said at least one collection tube is operably connected to said chamber by said connecting tube; and
an overflow tube, wherein said overflow tube is adapted to fit into at least one collection tube, further wherein said overflow tube discharges to the collection funnel or the reservoir.

13. The urination collection device of claim 12, wherein said valve is a turn valve.

14. The urination collection device of claim 12, wherein said valve is a pinch valve.

15. The urination collection device of claim 12, further comprising a multidirectional stopcock located between said connecting tube and said at least one collection tube.

16. The urination collection device of claim 15, further comprising two, three, or four collection tubes.

17. The urination collection device of claim 12, wherein said reservoir is selected from the group consisting of a bed pan, a toilet, and a collection bag.

18. The urination collection device of claim 15, further comprising a gel-like border on said collection funnel.

19. The urination collection device of claim 16, wherein a rim of said collection funnel is adapted to fit against the genitalia of a female patient.

20. The urination collection device of claim 12, wherein said urination collection device is constructed of a material that may be autoclaved.
